(19) United States
(12) Patent Application Publication
(10) Pub. No.: US 2021/0327065 A1
(43) Pub. Date: Oct. 21, 2021

(54) PROSTHESIS SCANNING AND IDENTIFICATION SYSTEM AND METHOD

(71) Applicant: Mark B. Wright, Rupert, ID (US)

(72) Inventor: Mark B. Wright, Rupert, ID (US)

(21) Appl. No.: 17/232,197

(22) Filed: Apr. 16, 2021

Related U.S. Application Data
(60) Provisional application No. 63/012,082, filed on Apr. 18, 2020.

(51) Int. Cl. G06T 7/00; G16H 70/00; G16H 50/70; G16H 30/40; G06N 3/08; G06N 7/00

(52) U.S. Cl. CPC G06T 7/0014; G16H 70/00; G16H 50/70; G06T 2207/10072; G06N 3/08; G06N 7/005; G06T 2207/30052; G16H 30/40

(57) ABSTRACT

The present application is directed to a Prosthesis Scanning and Identification System and Method used to positively identify an implanted prosthesis or any other implanted orthopedic device. The Prosthesis Scanning and Identification System and Method enables the positive identification of an implanted prosthesis or device by providing the steps of: (1) obtaining an initial conventional X-ray radiograph, or other suitable imaging of the affected area, and especially procuring the profile of an implanted prosthesis; (2) photographing the resulting X-ray radiograph then scanning said photograph using a smartphone and configured smartphone application; (3) searching a configured prosthesis identification database stored on a central server, accessible using a smartphone application, for profiles similar to the scanned prosthesis image; and (4) obtaining a list of probable prosthesis models based on the scanned images and profile comparisons.

### FIELD OF THE INVENTION

A system and method for scanning and identification of an implanted prosthesis or any other implanted orthopedic device is provided. More particularly, the Prosthesis Scanning and Identification System and Method of the present invention will be embodied in a smartphone application which will have the capabilities of photographing and scanning a conventional X-ray image or any other imaging format, then searching a prosthesis profile identification database to positively identify a prosthesis unit which has previously been implanted into a human joint. The Prosthesis Scanning and Identification System and Method enables the positive identification of an implanted prosthesis by providing the steps of: (1) obtaining an initial conventional x-ray radiograph, or other suitable imaging of the affected area, and especially procuring the profile of an implanted prosthesis; (2) photographing the resulting radiograph then scanning said photograph using a smartphone and configured smartphone application; (3) searching a configured prosthesis identification database, configured within a smartphone application, for profiles similar to the scanned prosthesis image; and (4) obtaining a list of possible prosthesis manufacturers and models in order of probability based on the analyzed scanned images.

### BACKGROUND OF THE INVENTION

There is growing need to provide a new and refined method of performing delicate surgical operations including hip, shoulder and knee replacements. The similarity in these operations is that the implants have to be inserted into a major bone in the area and when there is a problem with them, the previously implanted prosthesis with developed negative issues has to be removed.

As with any other mechanical device, a total hip replacement, or other implanted devices in other sites like the elbow, wrist, shoulder ankle or other, can be subject to various forms of mechanical or biological failure. For example, such a failure may require a revision of the hip replacement to address the cause of failure and its consequences. A revision of a total hip replacement is called a revision, total hip revision or any other implanted device revision, or femoral hip stem explant.

The revision hip implant is comprised of four parts that work together to restore the original function of the ball-and-socket joint. These four parts are as follows: (A) a metal hip stem that is inserted into the top of the truncated femur (thighbone); (B) a metal cup in the pelvis holds the cup liner; (c) a cup liner which holds the femoral head; and (D) the femoral head or ball which is attached to the top of the hip stem and is inserted into the liner to form the ball-and-socket joint.

The wearing down of the plastic component has an unfortunate side effect. The tiny plastic particles that wear off are attacked by your body's immune system, and this immune response also attacks the healthy bone around your implant. This leads to a condition called osteolysis, in which the bone in the area around the joint implant softens as it is absorbed by the body, thus making the implant unstable and in need of revision.

If the bone next to the primary implant is fractured in an accident, revision surgery may be required in order to provide a safe, stable joint. In this case, the original implant may need to be removed, the fracture addressed and a revision joint implanted.

In a low percentage of cases, the hip may become infected after surgery. Although it may be successfully treated with antibiotics, there are severe cases where a follow-up revision surgery may be required.

Hip revision operations are performed relatively infrequently. In the United States, there are approximately 18 revision hip replacements performed for every 100 hip replacements. The most common reasons for revision surgery are as follows: (A) repetitive (recurrent) dislocation of a hip replacement; (B) mechanical failure (implant wear and tear—loosening or breakage); and (C) infection.

Often, a surgeon will see a new patient in their office that has had joint replacement surgery done in other places. Years later the new patient comes in with issues surrounding the joint replacement surgery previously performed, where those issues need to be addressed by the new surgeon. It would be significantly helpful to the new surgeon to know, at that first visit, or early on in the treatment of those prosthesis implant issues, what was previously implanted in that patient. Therefore, it would be highly beneficial to the new patient and the new surgeon if the prosthesis could be positively identified before any revision surgery is attempted to correct the issues with that prosthesis presented by the new patient.

It would confer a great advantage on the surgeon if they knew before performing surgery on a patient, what prosthesis was implanted previously. There are numerous advantages in knowing before going in, what to expect. First, knowing what prosthesis was implanted gives the surgeon a heads up on what tools may be necessary to extract the prosthesis. It gives the surgeon time to order the correct parts that may be needed to correct the prosthesis issues. Information about when the prosthesis was manufactured, the shape and configuration of the prosthesis, and the known components of that particular prosthesis, would enable the surgeon to determine whether parts were available by contacting the manufacturer's representative to obtain more knowledge regarding that particular prosthesis.

Additionally, the correct identification of the previously implanted prosthesis would enable the surgeon to correctly identify which of a couple of options that might be needed to remove the implanted prosthesis in a revision surgery. Thus, the surgeon could more accurately order the proper parts and tools required to correct the issues surrounding a particular previously implanted prosthesis. By having the correct identification the surgeon could contact the manufacturer's representative and obtain critical information regarding the particular prosthesis device, including the history and efficacy of that device. Knowing what to expect, before initiating surgery, confers a significant advantage on the surgeon, and likewise, confers a great benefit on the patient knowing that the surgeon is armed with valuable information prior to treatment to correct the medical issues which have developed with that particular prosthesis, also if any device recalls are known.

Furthermore, the manufacturing companies would gain from the positive identification of their prosthesis products, in that they could monitor the success or failure rate of previously implanted devices which they brought to the marketplace. This may help those companies in future marketing efforts as well as research and development of new prosthesis devices.

Numerous innovations for post implant imaging have been provided in the prior art described as follows. Even though these innovations may be suitable for the specific individual purposes to which they address, they differ from the present Prosthesis Scanning and Identification System hereinafter contrasted. The following is a summary of those prior art patents most relevant to the Prosthesis Scanning and Identification System at hand, as well as a description outlining the difference between the features of the present application and those of the prior art.

In U.S. Pat. No. 8,995,731 of Joglekar the disclosure relates to image-based characterization of implanted medical leads used for electrical stimulation therapy. Characterization of implanted leads may include determination of lead configuration and lead orientation. The lead characterization techniques may make use of two-dimensional (2D) lead imaging in combination with known three-dimensional (3D) lead configuration data for various lead types. Lead characteristics determined from 2D lead imaging may be compared to lead dimensions calculated from known 3D lead characteristics to characterize implanted leads in terms of lead configuration and orientation. The lead characterization may be used to automatically determine or verify led configuration and orientation, and to aid in programming electrical stimulation therapy parameters.

This patent describes a method for image-based characterization of implanted medical lead for electrical stimulation therapy for the primary purpose of verifying lead configuration and orientation. It is not used for identification of particular implants such that a surgeon would know before initiating surgery what to expect after opening up the patient.

In U.S. Pat. No. 8,160,328 of Goetz et al. the disclosure is related to characterization of implanted electrical stimulation electrode arrays using post-implant imaging. The electrode arrays may be carried by implanted leads. Characterization of implanted electrode arrays may include identification of the type or types of leads implanted within a patient and/or determination of positions of the implanted leads or electrodes carried by the leads relative to one another or relative to anatomical structures within the patient. In addition, the disclosure relates to techniques for specifying or modifying patient therapy parameters based on the characterization of the implanted electrode arrays.

This is another patent that describes the characterization of implanted electrical stimulation electrode arrays using post-implant imaging wherein that characterization may include identification of the type of leads implanted within a patient. It cannot used for identification of particular prosthesis implants such as those used in joint replacement surgery, such that a surgeon would know what to expect before initiating surgery or “opening up” the patient to correct implant issues presented.

US Patent Application Publication No. 2010/0127075 of Flood describes a medical implant verification card (MIVC) having information provided on one or both sides thereof. Information for enabling access to a Medical Implant Verification Account (MIVA) of a patient is one the card. An image showing a medical implant as implanted within a body of the patient (e.g., reproduction of an x-ray image) is on the card. An image of an actual implant operation scar of the patient is on the card. The implant operation scar image shows a scar on the body of the patient resulting from implantation of the medical implant within the body of the patient. Implant identification information designating a type of the medical implant is on the card. Information designating a name of a surgeon having performed the operation for implanting the medical implant and/or information for contacting the surgeon is on the card.

This patent discloses the use of a medical implant verification card for enabling access to a Medical Implant Verification Account (MIVA) of a patient. While it contains an image of the external scar and designates the name of the surgeon performing the operation and that surgeons contact information, it does not allow for the positive identification of the implanted prosthesis.

U.S. Pat. No. 7,194,120 of Wicker et al. describes methods and computer systems for determining the placement of an implant in a patient in need thereof comprising the step of analyzing intensity-based medical imaging data obtained from a patient, isolating an anatomic site of interest from the imaging data, determining anatomic spatial relationships with the use of an algorithm, wherein the algorithm is optionally automated.

This patent describes a method for using a computer system for determining the placement of an implant in a patient, it does not enable identification of that implant which is the critical information required for a surgeon to correct medical issues surrounding the implant.

US Patent Application Publication No. 2017/0128027 of Nathaniel et al. describes a system for measuring the true dimensions and orientation of objects in a two dimensional image. The system is comprised of a ruler comprising at least one set of features each comprised of two or more markers that are identifiable in the image and having a known spatial relationship between them and a software package comprising programs that allow extension of the ruler and other objects in the two dimensional image beyond their physical dimensions or shape. The system can be used together with radiographic imagery means, processing means, and display means to take x-ray images and to measure the true dimensions and orientation of objects and to aid in the identification and location of a surgery tool vs. anatomy in those x-ray images. The invention provides a method of drawing and displaying on a two dimensional x-ray image measurements of objects visible in said image, graphical information, or templates of surgical devices.

This patent describes a system for making accurate measurements within two dimensional images, such as x-rays, and to aid in the identification and location of a surgery tool vs. anatomy in those x-ray images, it alone could not be used to positively identify implanted protheses as is the case for the present invention.

None of the foregoing prior art teaches or suggests the particular unique features of the Prosthesis Scanning and Identification System and thus clarifies the need for further improvements in the systems that can be used for these purposes. The surgical management of complications surrounding patients who have undergone hip arthroplasty necessitates accurate identification of the femoral implant manufacturer and model. Failure to do so risks delays in care, increased morbidity, and further economic burden. Because few arthroplasty experts can confidently classify implants using plain radiographs, automated image processing using artificial intelligence (AI), machine learning (ML), deep learning and regression analysis for implant identification offers an opportunity to significantly improve the value of care rendered.

In this respect, before explaining at least one embodiment of the Prosthesis Scanning and Identification System in detail it is to be understood that the design is not limited in its application to the details of construction and to the arrangement of the components set forth in the following description or illustrated in the drawings. The Prosthesis Scanning and Identification System is capable of other embodiments and of being practiced and carried out in various ways. Also, it is to be understood that the phraseology and terminology employed herein are for the purpose of description and should not be regarded as limiting.

### SUMMARY OF THE INVENTION

The principle advantage of the of the Prosthesis Scanning and Identification System is that it enables surgeons to identify an implanted prosthesis prior to taking corrective action.

Another advantage of using the Prosthesis Scanning and Identification System is that an implanted prosthesis can be positively identified using commonly available tools such as a smartphone and an X-ray or other radiographic device.

Another advantage of using the Prosthesis Scanning and Identification System is that once an implanted prosthesis is positively identified, a surgeon can evaluate problems with the implant and order the correct parts for taking corrective action.

Another advantage of using the Prosthesis Scanning and Identification System, a surgeon can obtain critical information about the implanted prosthesis model, such as the year of manufacture.

Another advantage of using the Prosthesis Scanning and Identification System, a surgeon can obtain the identified prosthesis manufacturer company information.

Another advantage of using the Prosthesis Scanning and Identification System, a surgeon can obtain the identified prosthesis manufacturer company representative information.

Another advantage of using the Prosthesis Scanning and Identification System, a surgeon can obtain the identified prosthesis manufacturer company representative contact information.

Another advantage of using the Prosthesis Scanning and Identification System is to have significantly less mortality and morbidity in revision surgery procedures, by knowing what to expect before going in.

Another advantage of using the Prosthesis Scanning and Identification System is to have a system utilizing a smartphone application and an on-line database, coupled with artificial intelligence (AI), machine learning (ML), deep learning and regression analysis to positively identify implanted protheses prior to surgery.

Finally, an advantage of the Prosthesis Scanning and Identification System, is that a surgeon will know what to expect before initiating revision surgery enabling improved surgical management of complications surrounding patients who have undergone hip arthroplasty, and thereby lessens the risks associated with delays in care, decreases morbidity, and minimizes further economic burden on the patient.

The Prosthesis Scanning and Identification System and Method enables the positive identification of an implanted prosthesis by providing the steps of: (1) obtaining an initial conventional x-ray radiograph, or other suitable imaging of the affected area, and especially procuring the profile of an implanted prosthesis; (2) photographing the resulting radiograph then scanning said photograph using a smartphone and configured smartphone application; (3) searching a configured prosthesis identification database, configured within a smartphone application, for prosthesis profiles similar to the scanned prosthesis image; and (4) obtaining a list of possible prosthesis manufacturers and models in order of probability based on the analyzed scanned images. Although all of the examples of medical imaging within the instant application involve X-ray radiography, it is anticipated that other modes of visualization of the implants may be used, including but not limited to X-ray computed tomography (CT), magnetic resonance imaging (MRI), computed axial tomography (CAT) scans, single-photon emission computed tomography (SPECT), thermography (heat mapping and pixel processing and analysis) and all other appropriate forms of imaging the implanted prothesis for identification purposes.

Initial imaging may be done of the anterior-posterior (AP) view only, the AP view and a Lateral view, as well as other views as necessary to scan and input the prosthesis profile into the matching database for searching potential hits on identification of the prosthesis by the obtained profile scans. A prosthesis profile matching database then computes the most likely identification matches, based on percentage of accuracy, by using an algorithm specifically configured and programmed to make comparisons of known prosthesis profiles within the database. The prosthesis profile matching database will be generated, updated and maintained on a central server, wherein all implant manufacturing companies will share data on each implant manufactured, the history of the implant, and other relevant data regarding the implant will be provided and entered into the database as required.

By using a single or multiple view scans, the database then makes potential matches lists based on the most likely match with the provided profile from the initial imaging. Additionally, the prosthesis matching database will generate a report to enable the surgeon to contact the implant manufacturing company, and will provide manufacturing company representative contact information for the convenience of the surgeon's office. Armed with the model and manufacturer information, the surgeon can perform additional research into the implant prior to surgery to correct issues with the identified implant.

With respect to the above description then, it is to be realized that the optimum dimensional relationships for the parts of the Prosthesis Scanning and Identification System and Method, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present design. Therefore, the foregoing is considered as illustrative only of the principles of the Prosthesis Scanning and Identification System and Method. Further, since numerous modifications and changes will readily occur to those skilled in the art, it is not desired to limit the Prosthesis Scanning and Identification System and Method to the exact construction and operation shown and described, and accordingly, all suitable modifications and equivalents may be resorted to, falling within the scope of this application.

While the system and method has or will be described for the sake of grammatical fluidity with functional explanations, it is to be expressly understood that the claims, unless expressly formulated under 35 USC 112, or similar applicable law, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112, or similar applicable law. The Prosthesis Scanning and Identification System and Method can be better visualized by turning now to the following drawings wherein like elements are referenced by like numerals.

### BRIEF DESCRIPTION OF THE DRAWINGS

The accompanying drawings, which are incorporated in and form a part of this specification, illustrate embodiments of the Prosthesis Scanning and Identification System and Method and together with the description, serve to explain the principles of this application.

FIG. 1 depicts a conventional x-ray taken from the front to back of a patient resulting in a radiograph of the anterior-posterior (AP) view of the patient's hip area, showing an implanted prosthesis.

FIG. 2 depicts a conventional x-ray taken from the side of a patient resulting in a radiograph of the Lateral view of the patient's hip area, showing an implanted prosthesis.

FIG. 3 depicts a radiograph resulting from the x-ray taken in FIG. 1, showing an implanted prosthesis from the AP view.

FIG. 4 depicts a smartphone camera photograph of the radiograph resulting from the x-ray taken in FIG. 1, showing an implanted prosthesis from the AP view.

FIG. 5 depicts a smartphone application scan of the profile of the implanted prosthesis shown in the smartphone camera photograph of the radiograph of FIG. 4 from the AP view.

FIG. 6 depicts a preliminary generated prosthesis database report of possible identification matches for the AP view scan of the profile of the implanted prosthesis shown in FIG. 5 from the AP view.

FIG. 7 depicts a radiograph resulting from the x-ray taken in FIG. 2, showing an implanted prosthesis from the Lateral view.

FIG. 8 depicts a smartphone camera photograph of the radiograph resulting from the x-ray taken in FIG. 2, showing an implanted prosthesis from the Lateral view.

FIG. 9 depicts a smartphone application scan of the profile of the implanted prosthesis shown in the smartphone camera photograph of the radiograph of FIG. 8 from the Lateral view.

FIG. 10 depicts a preliminary generated prosthesis database report of possible identification matches for the scan of the profile of the implanted prosthesis shown in FIG. 9 from the Lateral view.

FIG. 11 depicts a flow chart of the prosthesis identification database analysis of the AP view radiograph scans and the Lateral view radiograph scans to determine an implant database match and to generate a report on the identified implant including information on the manufacturer of the identified implant.

FIG. 12 depicts one type of prosthesis stem implant having a ball, collar and orifices within the stem.

FIG. 13 depicts another type of prosthesis stem implant having a trunnion with no ball, a collar and a characteristic stem end modification.

FIG. 14 depicts a prosthesis stein implant having a trunnion with no ball, porous upper section having orifices therein, and fluted grooves within the stem.

FIG. 15 depicts a prosthesis stem implant having a trunnion with no ball, porous upper section having orifices therein, and a shortened stem length.

FIG. 16 depicts a prosthesis stem implant having a trunnion with no ball, a porous upper and lower stem having a shortened stem length.

FIG. 17 depicts a prosthesis stem implant having a ball, and a barbed stem end.

FIG. 18 depicts a prosthesis stem implant having a ball, a sectioned trunnion, a sectioned collar and a long smooth stem.

FIG. 19 depicts a prosthesis stem implant having a ball, an extended collar and smooth stem.

FIG. 20 depicts a prosthesis stem implant having a ball, a porous trunnion and a porous stem.

FIG. 21 depicts a prosthesis stem implant having a ball, curved collar and a smooth shortened stem.

FIG. 22 depicts a prosthesis stem implant having a ball, a porous ball cup, a porous upper stem section and a fluted stem.

FIG. 23 depicts a prosthesis stem implant having a ball, a porous ball cup, a porous upper stem section and a shortened stem.

FIG. 24 depicts a prosthesis stem implant having no ball, a trunnion with orifice therein, a porous stem, and orifices with the porous stem.

FIG. 25 depicts a prosthesis stem implant having a trunnion, a porous stem and a fluted upper stem section.

FIG. 26 depicts a prosthesis stem implant having a trunnion and a porous tapered stem.

FIG. 27 depicts a prosthesis stem implant having a sectioned trunnion, a porous upper stem section and a large diameter smooth lower stem section.

FIG. 28 depicts a trunnion and an upper stem section having an orifice therein.

FIG. 29 depicts a prosthesis stem implant having a sectioned trunnion, a collar and an upper stem section having a plurality of characteristic orifices therein.

FIG. 30 depicts a prosthesis stem implant having a sectioned trunnion, a fish-hook shaped collar and a long smooth stem.

FIG. 31 depicts a prosthesis stem implant having a sectioned trunnion, no collar and a long smooth stem.

### DETAILED DESCRIPTION OF THE PREFERRED EMBODIMENTS

As required, the detailed embodiments of the present Prosthesis Scanning and Identification System and Method 10A and 10B are disclosed herein, however, it is to be understood that the disclosed embodiments are merely exemplary of the design that may be embodied in various forms. Therefore, specific functional and structural details disclosed herein are not to be interpreted as limiting, but merely as basic for the claims and as a representative AP view basis for teaching one skilled in the art to variously employ the present design in virtually any appropriately detailed structure.

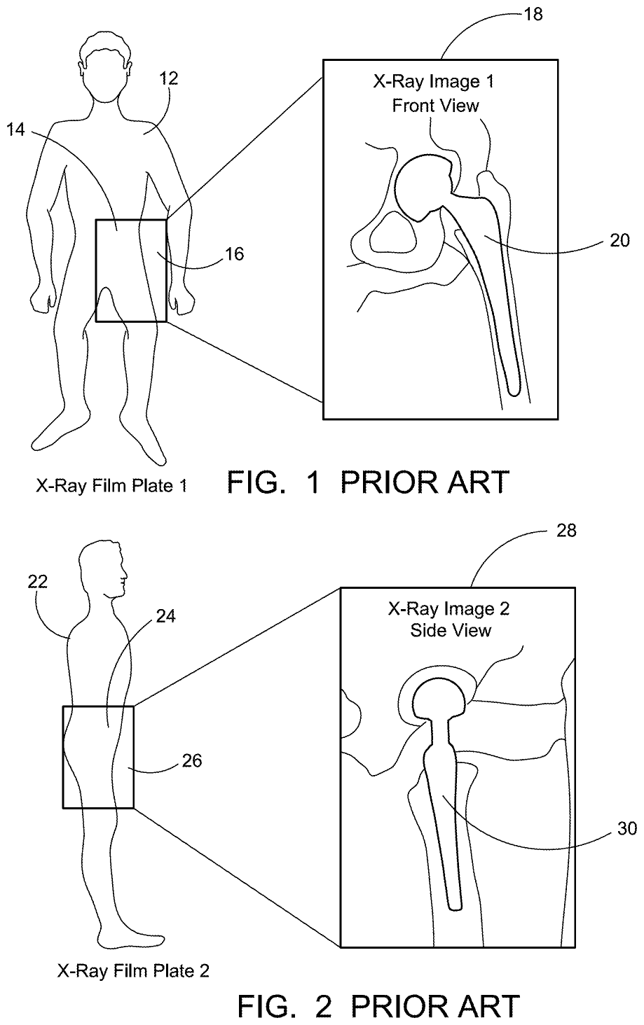

FIG. 1 depicts a conventional x-ray film plate 16 taken from the front of a patient 12 facing the source of x-rays, resulting in a radiograph 18 of the AP view of the patient's hip area 14, showing a profile of an implanted prosthesis 20. The x-ray radiograph may be taken of the front view of the patient 12 as shown here, or the rear view of the patient 12, in the affected area, as long as the prosthesis front or rear profile 20 is captured.

FIG. 2 depicts a conventional x-ray film plate 26 taken from the right side 24 of the same patient this time facing 90 degrees from the source of x-rays 22 resulting in an X-ray radiograph 28 of the right Lateral view of the patient's hip area 24, showing a profile of an implanted prosthesis 30. The x-ray radiograph may be taken of the right Lateral view of the patient 22 as shown here, or the left Lateral view of the patient 22, in the affected area, as long as the prosthesis side profile 30 is captured.

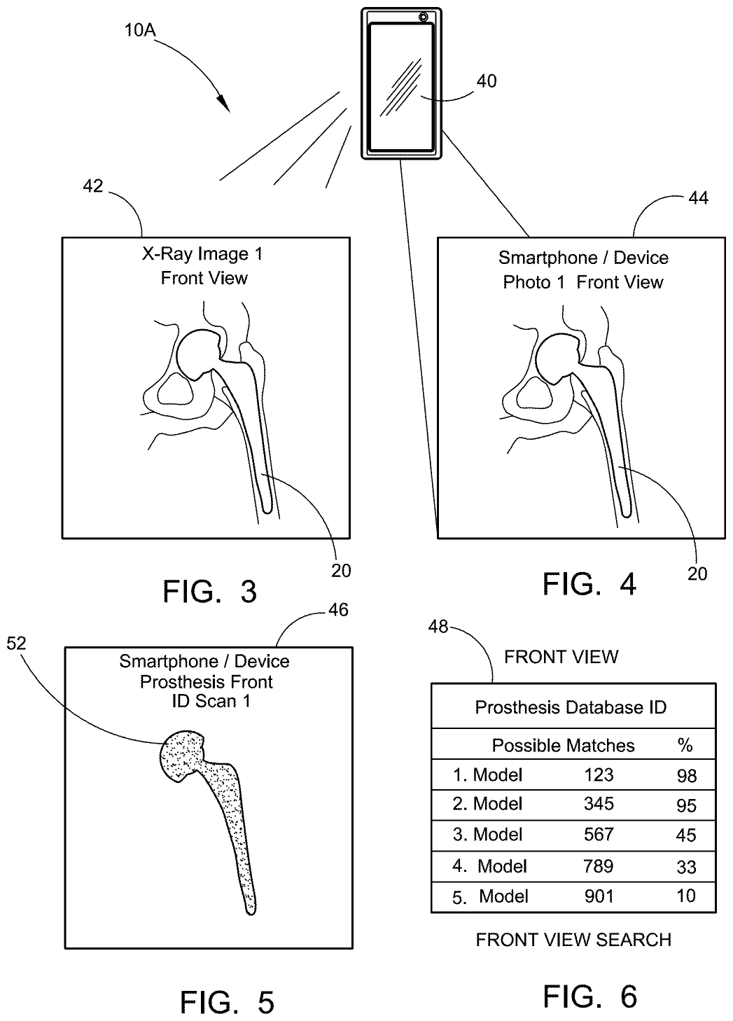

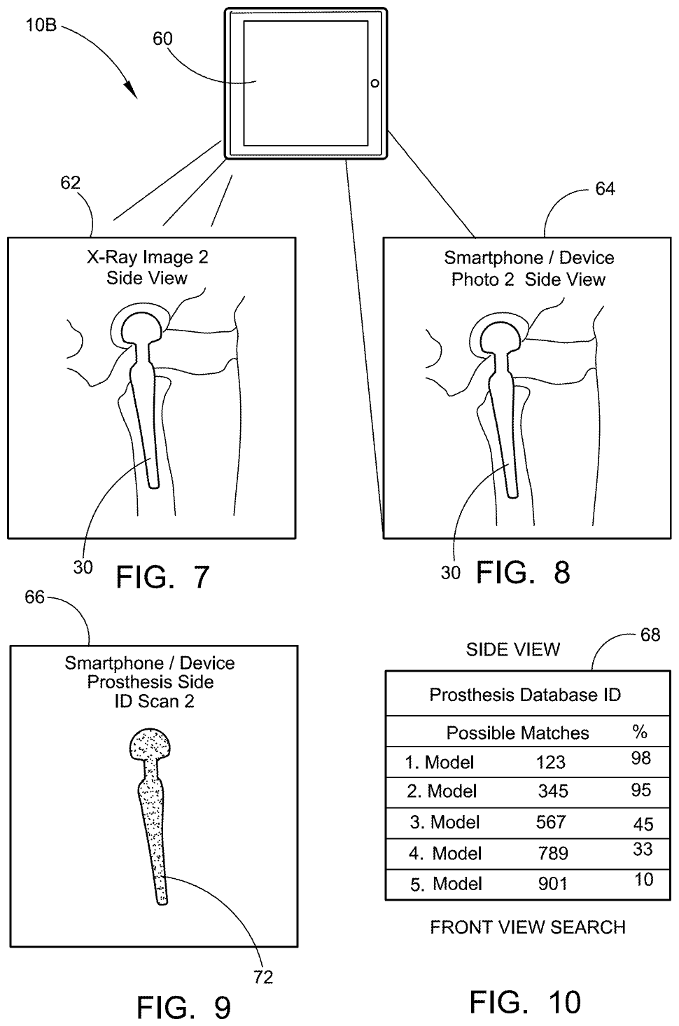

FIG. 3 depicts an X-ray radiograph resulting from the x-ray taken in FIG. 1, showing an implanted prosthesis from the AP view. Having obtained an X-ray radiograph 42, an X-ray radiographic image of the front or rear view of the patient's affected area, the Prosthesis Scanning and Identification System and Method 10A now begins the process of identification of the implanted prosthesis from the resulting profile 20. A smartphone 40 (or a tablet computer may be used, see FIGS. 7 and 8 below) equipped with a camera capable of photographing the radiograph 42, generates a photograph 44 of the X-ray radiograph 42. A smartphone application configured to scan and identify implanted protheses then scans the resulting photograph 44 of the radiograph 42 and generates a scanned image 46 of the implanted prosthesis 20 resulting in a detailed prosthesis profile scan 52 for comparison by the prosthesis profile identification program and database, previously configured and stored on a database server, associated with the smartphone application.

FIG. 4 depicts a smartphone 40 camera photograph 44 of the X-ray radiograph 42 resulting from the X-ray taken in FIG. 1, showing an implanted prosthesis profile 20 from the AP view. A smartphone 40 (or a tablet computer may be used, see FIGS. 7 and 8 below) equipped with a camera capable of photographing the X-ray radiograph 42, generates a photograph 44 of the X-ray radiograph 42. This photograph contains the front/rear view of the prosthesis profile 20 for subsequent analysis and identification.

FIG. 5 depicts a smartphone application scan of the profile of the implanted prosthesis shown in the smartphone camera photograph of the radiograph of FIG. 4 from the AP view. A smartphone 40 application configured to scan and identify implanted protheses then scans the resulting photograph 44 of the X-ray radiograph 42 and generates a scanned image 46 of the implanted prosthesis 20 resulting in a detailed prosthesis profile scan 52 for comparison by the prosthesis profile identification program and database, previously configured and stored on a database server, associated with the smartphone application. The smartphone application analyzes the front/rear scan prosthesis profile 52 and compares it to know protheses profiles in the prosthesis profile matching database. This analysis results in a generated report of possible front prosthesis profile matches, with percentage confidence, in order of a likelihood of the positive match (see FIG. 6).

FIG. 6 depicts a preliminary generated prosthesis front/rear profile database report 48 of possible prosthesis profile identification matches for the front/rear view scan of the profile of the implanted prosthesis shown in FIG. 5 from the AP view. The generated prosthesis front/rear profile database report 48 lists the possible prosthesis model matches, along with the percentage confidence of the profile match. The models are listed by manufacturer model numbers found within the prosthesis profile matching database. The front/rear prosthesis profile may be all that is required to make a high confidence match. If more data is required, then a Lateral view x-ray and profile may be generated for scanning and further matching analysis by the prosthesis matching database and smartphone application system (see FIG. 7 below).

FIG. 7 depicts a radiograph 62 resulting from the x-ray taken in FIG. 2, showing an implanted prosthesis profile 30 from the Lateral view. Having obtained an X-ray radiograph 62, an x-ray image of the right side or left Lateral view of the patient's affected area, the Prosthesis Scanning and Identification System and Method 10B now begins the process of identification of the implanted prosthesis from the resulting profile 30. A tablet computer 60 (or a smartphone may be used, see FIGS. 3 and 4 above) equipped with a camera capable of photographing the radiograph 62, generates a photograph 64 of the radiograph 62. A smartphone application configured to scan and identify implanted protheses then scans the resulting photograph 64 of the radiograph 62 and generates a scanned image 66 of the implanted prosthesis 30 resulting in a detailed prosthesis profile scan 72 for comparison by the prosthesis profile identification program and database, previously configured and stored on a database server, associated with the smartphone application.

FIG. 8 depicts a tablet computer 60 camera photograph 64 of the radiograph 62 resulting from the x-ray taken in FIG. 2, showing an implanted prosthesis profile 30 from the Lateral view. A tablet computer 60 (or a smartphone may be used, see FIGS. 3 and 4 below) equipped with a camera capable of photographing the radiograph 62, generates a photograph 64 of the radiograph 62. This photograph 64 contains the right/left view of the prosthesis profile 30 for subsequent analysis and identification. Although all of the examples of medical imaging within the instant application involve X-ray radiography, it is anticipated that other modes of visualization of the implants may be used, including but not limited to X-ray computed tomography (CT), magnetic resonance imaging (MRI), computed axial tomography (CAT) scans, single-photon emission computed tomography (SPECT), thermography (heat mapping and pixel processing and analysis) and all other appropriate forms of imaging the implanted prothesis for identification purposes.

FIG. 9 depicts a smartphone application scan of the profile of the implanted prosthesis shown in the tablet computer camera photograph of the radiograph of FIG. 2 from the Lateral view. A tablet computer (or smartphone) 60 application configured to scan and identify implanted protheses then scans the resulting photograph 64 of the radiograph 62 and generates a scanned image 66 of the implanted prosthesis 30 resulting in a Lateral view detailed prosthesis profile scan 72 for comparison by the prosthesis profile identification program and prosthesis matching database, previously configured and stored on a database server, associated with the tablet/smartphone application. The tablet/smartphone application analyzes the Lateral view scan prosthesis profile 72 and compares it to know protheses profiles in the prosthesis profile matching database. This analysis results in a generated report of possible front prosthesis profile matches, with percentage confidence, in order of a likelihood of the positive match (see FIG. 10).

FIG. 10 depicts a preliminary generated prosthesis right/left side profile database report 68 of possible prosthesis profile identification matches for the Lateral (side) view scan of the profile of the implanted prosthesis shown in FIG. 9 from the Lateral view. The generated report lists the possible prosthesis model matches, along with the percentage confidence of the prosthesis model profile match. The models are listed by manufacturer model numbers found within the prosthesis profile matching database. The Lateral view prosthesis profile may be all that is required to make a high confidence match. If more data is required, then an AP view x-ray and profile may be generated for further accuracy in the matching analysis performed by the prosthesis matching database and tablet/smartphone application system (see FIGS. 3-6 above).

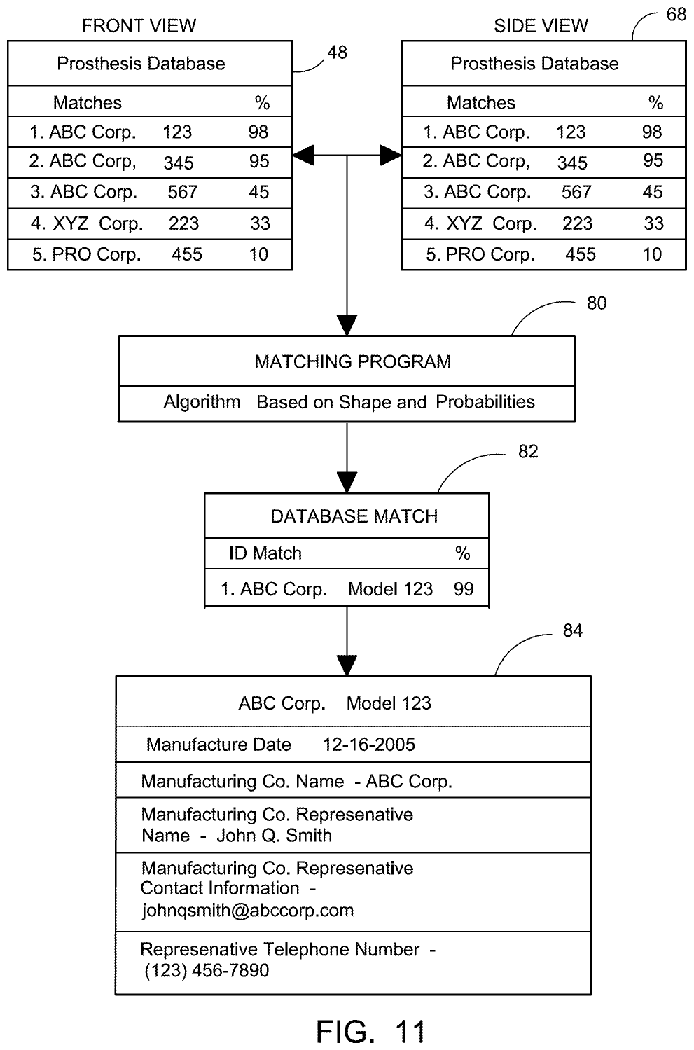

FIG. 11 depicts a flow chart of the Prosthesis Scanning and Identification System and Method 10A and 10B for prosthesis identification. Database analysis of the AP view radiograph scans 48 and database analysis of the Lateral view radiograph scans 68, previously generated in FIGS. 1-10 above, are analyzed to determine an implant database match and to generate a report on the identified implant including information on the manufacturer of the identified implant. AP view prosthesis matching database analysis 48 and Lateral view prosthesis matching database are fed into a matching program 80 configured to perform matching operations using a matching algorithm based on implant profile shape, and generates possible matched based on probabilities of match accuracy. The matching program 80, located in memory on a central server, uses artificial intelligence (AI), machine learning (ML) deep learning and regression analysis to generate a database match report 82 indicating the most likely identification of the implant scanned in the previously performed process of imaging and scanning the prosthesis implant profiles. Once a match has been calculated employing image pixel processing comparisons, a full report 84 on the identified implant manufacturer and model is then generated, by the central server, including central server stored information regarding the manufacturing date, the manufacturing company, the manufacturing company representative name, manufacturing company representative contact information, including manufacturing company representative e-mail and telephone number. The surgeon can then contact the manufacturing company representative directly to obtain further information on the particular implant identified prior to surgery or other treatment. This information is invaluable to the surgeon as the knowledge of what to expect, before initiating surgery, as far as an implant manufacturer and model is concerned, makes ordering the correct parts and tools more efficient and can lead to better medical outcomes.

Artificial intelligence (AI) is a broad term referring to the application of computational algorithms that can analyze large data sets to classify, predict, or gain useful inferences in solving problems. Machine learning (ML) is a subset of AI that involves using real-world data sets to predict or estimate an outcome. These real-world data sets encompass “training sets” that the machine is able to study and “learn” from using pattern recognition. The training data set is then compared with a test data set that quantifies the accuracies of the aforementioned inferences for further calibration. Deep learning employs sophisticated algorithms that require little or no human supervision to analyze, calibrate, and provide inferences. These sophisticated algorithms include deep neural network models. It is anticipated that the present system will utilize smartphone application software and an on-line/cloud-base database, coupled with artificial intelligence (AI), machine learning (ML), deep learning and regression analysis to positively identify implanted protheses prior to surgery.

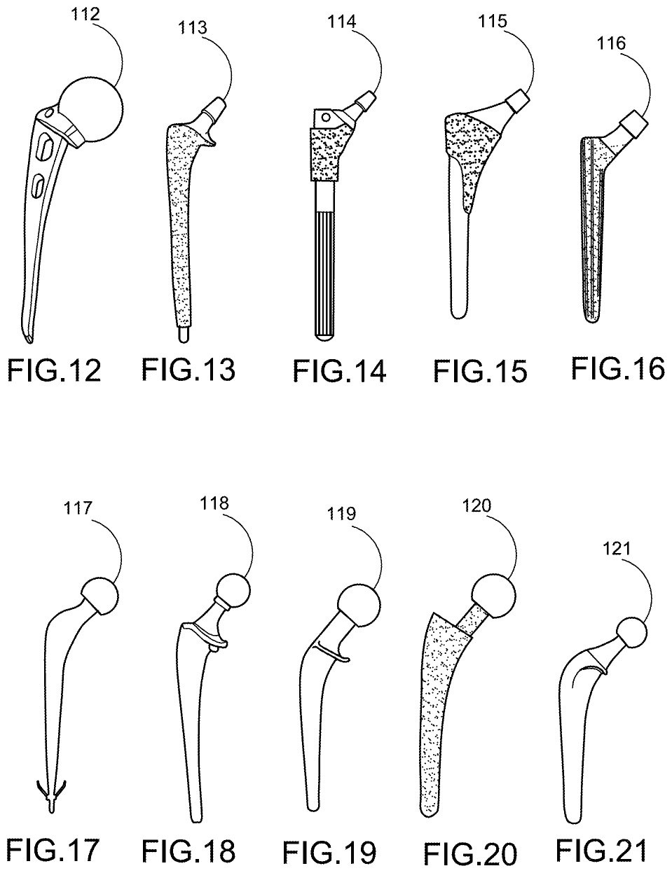

As an illustration of the varying and many configurations of possible implants, the femur stem component of a hip replacement is used herein as an example. In this regard, FIGS. 12-31 represent possible configurations and styles and shapes of the many implants found on the market. All of these previously manufactured, marketed and sold implants would be scanned for profiles, cataloged, and all of the information necessary for surgeons would be inputted into a central server's memory storage database to be used as the prosthesis profile matching database. This implant information would then be used to match prosthesis profiles and generate informational reports regarding the potential likely matches gleaned from the scans of images of the prostheses profiles. The prosthesis matching database would be made accessible, through the use of a downloadable smartphone or tablet computer application, to surgeons using the Prosthesis Scanning and Identification System and Method 10A and 10B for prosthesis identification. The following figures are meant to illustrate the various sizes and shapes of just one type of prosthesis which would be anticipated to be scanned and analyzed by the prosthesis profile matching database. Information regarding manufacturing would also be inputted into the central server for the purpose of generating informational reports on the possible matches.

FIG. 12 depicts one type of prosthesis stem implant 112 having a ball, collar and orifices within the stem.

FIG. 13 depicts another type of prosthesis stem implant 113 having a trunnion with no ball, a collar and a characteristic stem end modification.

FIG. 14 depicts a prosthesis stem implant 114 having a trunnion with no ball, porous upper section having orifices therein, and fluted grooves within the stem.

FIG. 15 depicts a prosthesis stem implant 115 having a trunnion with no ball, porous upper section having orifices therein, and a shortened stem length.

FIG. 16 depicts a prosthesis stein implant 116 having a trunnion with no ball, a porous upper and lower stem having a shortened stem length.

FIG. 17 depicts a prosthesis stem implant 117 having a ball, and a barbed stem end.

FIG. 18 depicts a prosthesis stem implant 118 having a ball, a sectioned trunnion, a sectioned collar and a long smooth stem.

FIG. 19 depicts a prosthesis stem implant 119 having a ball, an extended collar and smooth stem.

FIG. 20 depicts a prosthesis stem implant 120 having a ball, a porous trunnion and a porous stem.

FIG. 21 depicts a prosthesis stem implant 121 having a ball, curved collar and a smooth shortened stem.

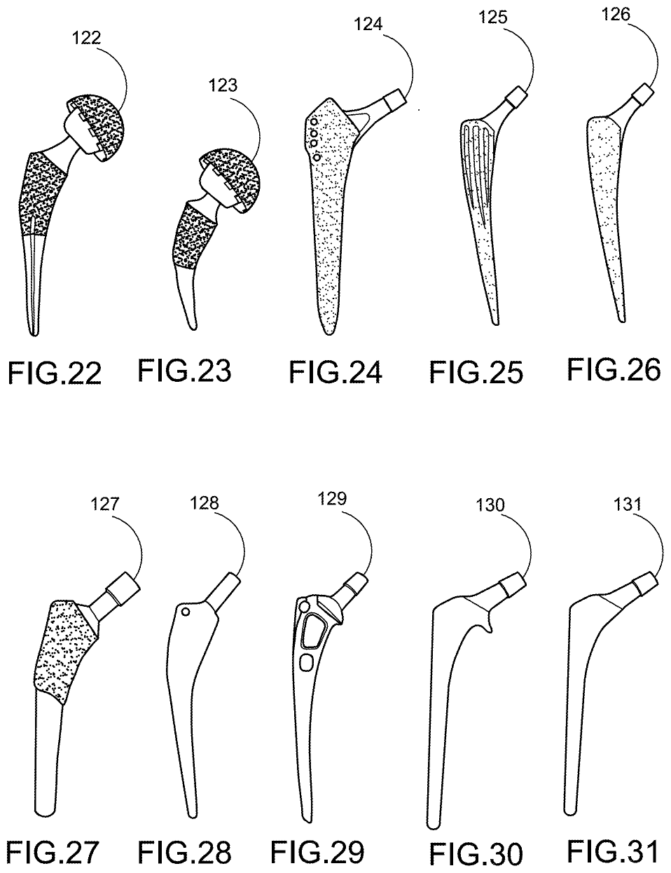

FIG. 22 depicts a prosthesis stem implant 122 having a ball, a porous ball cup, a porous upper stem section and a fluted stem.

FIG. 23 depicts a prosthesis stem implant 123 having a ball, a porous ball cup, a porous upper stem section and a shortened stem.

FIG. 24 depicts a prosthesis stem implant 124 having no ball, a trunnion with orifice therein, a porous stem, and orifices with the porous stem.

FIG. 25 depicts a prosthesis stem implant 125 having a trunnion, a porous stem and a fluted upper stem section.

FIG. 26 depicts a prosthesis stem implant 126 having a trunnion and a porous tapered stem.

FIG. 27 depicts a prosthesis stem implant 127 having a sectioned trunnion, a porous upper stem section and a lame diameter smooth lower stem section.

FIG. 28 depicts a prosthesis stem implant 128 having a trunnion and an upper stem section having an orifice therein.

FIG. 29 depicts a prosthesis stem implant 129 having a sectioned trunnion, a collar and an upper stem section having a plurality of characteristic orifices therein.

FIG. 30 depicts a prosthesis stem implant 130 having a sectioned trunnion, a fish-hook shaped collar and a long smooth stem.

FIG. 31 depicts a prosthesis stem implant 131 having a sectioned trunnion, no collar and a long smooth stem.

The key to the functioning of the Prosthesis Scanning and Identification System and Method 10A and 10B is the smartphone or tablet computer application. It is anticipated that the smartphone or tablet computer application described above may be administered on one or more central servers and the information transfer may be implemented by a global computer network, the Internet and/or a cloud-based server system. Such hardware, software or firmware applications may be implemented in the same device or within separate devices to support the various operations described in this disclosure.

When implemented in application software, the functionality ascribed to the systems, devices and techniques described in this disclosure may be embodied as instructions on a computer-readable medium such as random access memory (RAM), read-only memory (ROM), non-volatile random access memory (NVROM), electronically erasable programmable read-only memory (EPROM), FLASH memory, magnetic storage media, optical data storage media, or the like. It is anticipated that the present system will utilize smartphone application software and an on-line/cloud-base database, coupled with artificial intelligence (AI), machine learning (ML), deep learning and regression analysis to positively identify implanted protheses prior to surgery.

The Prosthesis Scanning and Identification System and Method 10A and 10B shown in the drawings and described in detail herein disclose arrangements of elements of particular construction and configuration for illustrating preferred embodiments of structure and method of operation of the present application. It is to be understood, however, that elements of different construction and configuration and other arrangements thereof, other than those illustrated and described may be employed for providing the Prosthesis Scanning and Identification System and Method 10A in accordance with the spirit of this disclosure, and such changes, alternations and modifications as would occur to those skilled in the art are considered to be within the scope of this design as broadly defined in the appended claims.

While certain embodiments of the Prosthesis Scanning and Identification System and Method have been described, these embodiments have been presented by way of example only, and are not intended to limit the scope of the disclosure. Indeed, the novel methods and systems described herein may be embodied in a variety of other forms. Furthermore, various omissions, substitutions and changes in the systems and methods described herein may be made without departing from the spirit of the disclosure. For example, one portion of one of the embodiments described herein can be substituted for another portion in another embodiment described herein. The accompanying claims and their equivalents are intended to cover such forms or modifications as would fall within the scope and spirit of the disclosure. Accordingly, the scope of the present inventions is defined only by reference to the appended claims.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

Further, the purpose of the foregoing abstract is to enable the U.S. Patent and Trademark Office, foreign patent offices worldwide and the public generally, and especially the scientists, engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

I claim:

1. A system for implanted prosthesis imaging, scanning and identification, comprising:
(a) a central server configured to store information regarding medical implants on a database populated with medical implant information including physical size, dimensions and shape profiles of medical implants;
(b) an image processing smartphone application configured to generate photographs, scanned images and profiles of medical implants;
(c) said image processing smartphone application further configured to process said scanned images and profiles of medical implants and send data to said central server to compare said implant profiles with known implant profiles stored on said central server; and
(d) wherein said smartphone application in combination with said central server generates lists of possible implant matches derived from said comparison of the scanned implant profile with known implant profiles stored on the central server.

2. The system for implanted prosthesis imaging, scanning and identification, according to claim 1 wherein said central server configured to store information regarding medical implants is accessible via a global computer network, the Internet and/or a cloud-based server network.

3. The system for implanted prosthesis imaging, scanning and identification, according to claim 1 wherein said image processing smartphone application further configured to process said scanned images and profiles of medical implants accesses said central server configured to store information regarding medical implants via a global computer network, the Internet and/or a cloud-based server network.

4. The system for implanted prosthesis imaging, scanning and identification, according to claim 1 wherein said image processing smartphone application further configured to process said scanned images and profiles of medical implants first photographs images of implants to be identified, scans said photographs, then sends said scans to said central server.

5. The system for implanted prosthesis imaging, scanning and identification, according to claim 1 wherein said a central server configured to store information regarding medical implants on a database populated with medical implant information utilizes artificial intelligence, machine leaning, deep learning and regression analysis algorithms to compare known implant profiles with said scans of implant images generated on said smartphone application and sent to said central server.

6. The system for implanted prosthesis imaging, scanning and identification, according to claim 1 wherein said image processing smartphone application further configured to process said scanned images and profiles of medical implants is capable of processing X-ray radiographs, X-ray computed tomography, magnetic resonance imaging, computed axial tomography scans, single-photon emission computed tomography, thermography, heat mapping and pixel processing images of the implanted prothesis for identification purposes.

7. The system for implanted prosthesis imaging, scanning and identification, according to claim 1 wherein anterior-posterior image views of the implant to be identified are analyzed.

8. The system for implanted prosthesis imaging, scanning and identification, according to claim 1 wherein said lateral image views of the implant to be identified are analyzed.

9. The system for implanted prosthesis imaging, scanning and identification, according to claim 1 wherein said smartphone application in combination with said central server generates lists of possible implant matches derived from said comparison of the scanned implant profile with known implant profiles stored on the central server, further includes generating probabilities of matches with known implant profiles stored on said central server.

10. The system for implanted prosthesis imaging, scanning and identification, according to claim 1 wherein said central server in combination with said smartphone application generates information on the manufacturer, model of implant, and contact information for obtaining further information from the manufacturer.

11. A method for making a system for implanted prosthesis imaging, scanning and identification, comprising the steps of:
(a) providing a central server configured to store information regarding medical implants, and populated with medical implant information including physical size and shape profiles of medical implants;
(b) providing an image processing smartphone application configured to generate photographs, scanned images and profiles of medical implants;
(c) configuring said image processing smartphone application to further process said scanned images and generate processed implant profiles of medical implants;
(d) comparing smartphone application processed implant profiles with known implant profile information stored on said central server; and
(d) generating lists of possible implant matches derived from said comparison of the scanned implant profile with known implant profiles stored on the central server.

12. The method for making a system for implanted prosthesis imaging, scanning and identification, according to claim 11, wherein said central server configured to store information regarding medical implants is accessible via a global computer network, the Internet and/or a cloud-based server network.

13. The method for making a system for implanted prosthesis imaging, scanning and identification, according to claim 11, wherein said image processing smartphone application further configured to process said scanned images and profiles of medical implants accesses said central server configured to store information regarding medical implants via a global computer network, the Internet and/or a cloud-based server network.

14. The method for making a system for implanted prosthesis imaging, scanning and identification, according to claim 11, wherein said image processing smartphone application further configured to process said scanned images and profiles of medical implants first photographs images of implants to be identified, scans said photographs, then sends said scans to said central server.

15. The method for making a system for implanted prosthesis imaging, scanning and identification, according to claim 11, wherein said a central server configured to store information regarding medical implants on a database populated with medical implant information utilizes artificial intelligence, machine leaning, deep learning and regression analysis algorithms to compare known implant profiles with said scans of implant images generated on said smartphone application and sent to said central server.

16. The method for making a system for implanted prosthesis imaging, scanning and identification, according to claim 11, wherein said image processing smartphone application further configured to process said scanned images and profiles of medical implants is capable of processing X-ray radiographs, X-ray computed tomography, magnetic resonance imaging, computed axial tomography scans, single-photon emission computed tomography, thermography, heat mapping and pixel processing images of the implanted prothesis for identification purposes.

17. The method for making a system for implanted prosthesis imaging, scanning and identification, according to claim 11, wherein anterior-posterior image views of the implant to be identified are analyzed.

18. The method for making a system for implanted prosthesis imaging, scanning and identification, according to claim 11, wherein said lateral image views of the implant to be identified are analyzed.

19. The method for making a system for implanted prosthesis imaging, scanning and identification, according to claim 1 wherein said smartphone application in combination with said central server generates lists of possible implant matches derived from said comparison of the scanned implant profile with known implant profiles stored on the central server, further includes generating probabilities of matches with known implant profiles stored on said central server.

20. A method for using a system for implanted prosthesis imaging, scanning and identification, comprising the steps of:
(a) providing a central server configured to store information regarding medical implants, and populated with medical implant information including physical size and shape profiles of medical implants;
(b) providing an image processing smartphone application configured to generate photographs, scanned images and profiles of medical implants;
(c) generating an X-ray radiograph anterior-posterior view image of an implanted prosthesis;
(d) generating an X-ray radiograph lateral view image of an implanted prosthesis;
(e) photographing said X-ray radiograph anterior-posterior view image;
(f) photographing said X-ray radiograph lateral view image;
(g) scanning said photograph of said X-ray radiograph anterior-posterior view image, thereby generating anterior-posterior view scan data for pixel processing;
(h) scanning said photograph of said X-ray radiograph lateral view image, thereby generating lateral view scan data for pixel processing;
(i) sending said anterior-posterior view scan data and sending said lateral view scan data then comparing said anterior-posterior view scan data and said lateral view scan data with said medical implant profile information populated on said central server; and
(j) generating a list of probable prosthesis models identified based on the scanned images.
